(19) United States
(12) Patent Application Publication
(10) Pub. No.: US 2021/0131972 A1
(43) Pub. Date: May 6, 2021

(54) METHODS AND MATERIALS FOR MERCURY DETECTION AND REMOVAL

(71) Applicant: University of Ontario Institute of Technology, Oshawa (CA)

(72) Inventors: Olena V. Zenkina, Oshawa (CA); Iraklii I. Ebralidze, Oshawa (CA); Fedor Naumkin, Toronto (CA); Jacquelyn Egan, Whitby (CA)

(21) Appl. No.: 16/673,072

(22) Filed: Nov. 4, 2019

(51) Int. Cl. G01N 21/78; C07D 417/14; C07D 409/14; C02F 1/28; C02F 1/48; B03C 1/01

(52) U.S. Cl. CPC G01N 21/78; C07D 417/14; C07D 409/14; C02F 2101/20; C02F 1/48; B03C 1/01; C02F 1/285

(57) ABSTRACT

Composite materials for the detection of analytes are described herein. The composite material includes a ligand-functionalized monolayer and a support material coupled to the ligand-functionalized monolayer. Methods of fluorescently detecting analytes and removing analytes from a solution are also described.

### FIELD

This disclosure relates generally to methods and materials for detecting analytes, and more specifically to methods and materials for detecting mercury.

### BACKGROUND

Detection of mercury (II) ions is a very important challenge facing modern society. Mercury is hardly biodegradable and an extremely prevalent toxic metal ion occurring in various natural and anthropogenic sources. Upon entering into aqueous systems, mercury (II) ions can be transformed by bacteria to a higher toxicity form, neurotoxic organic mercury, that then enter and accumulate in the food chain of ecological systems.

The development of effective methods and materials for detecting and differentiating mercury ions from other trace metal elements as well as designing and manufacturing of systems capable of selective capturing and removal of mercury ions from the media of interest are globally recognized priorities. In Canada and the US, the need for proper control on mercury content in the environment is crucial due to the largest in the world system of great lakes, bearing 21% of world fresh water sources. Contamination of water by mercury results in the accumulation of the most toxic organo-mercury compounds in the body of fish that very quickly transfer to animals and/or humans.

Soil contamination by mercury is another serious problem that needs to be taken into account. Plants easily absorb and accumulate mercury and as a result, the plant products from the contaminated areas contain a significant source of neurotoxic mercury.

In addition, synthetic materials and chemicals for pharmaceutical, cosmetic and food industries could be artificially contaminated by mercury ions during the synthetic process of materials production. Mercury poisoning results in devastating health effects (e.g. severe neurological problems and birth defects) for the population.

The development of an effective and safe methodology that provides fast and selective detection and removal of mercury ions from sources of various nature is important for environmental protection and cost-effective production of fine chemicals. For instance, the maximum acceptable concentration of 0.001 mg/L (1 mg/L) of mercury in drinking water has been established and allowed in Canada.

The amount of dissolved mercury in water is normally determined by cold vapor atomic absorption spectroscopy. This method requires bulky, not portable, and expensive equipment and highly qualified personnel to operate this equipment and prepare samples.

### SUMMARY

In accordance with a broad aspect, there is provided a composite material for the detection of mercury. The composite material includes a ligand-functionalized monolayer and a support material coupled to the ligand-functionalized monolayer. The ligand-functionalized monolayer includes one or more ligands having the following formula:

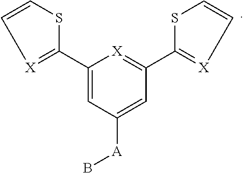

In the preceding formula, A comprises a linear or a cyclic aliphatic moiety, an aromatic ring or a fused aromatic ring system, a heteroaromatic ring, a fused heteroaromatic ring system, quaternary ammonium salt, or a combination thereof; B comprises hydrogen or a chemically derivatizable group such as alkene, alkyne, amino acid, azide, phosphate, phosphonate, carboxyl group, silane, siloxane, sulfate, quaternary ammonium salt, thiol, alkyl thiol, or thioester; and X comprises carbon, nitrogen, sulphur or oxygen.

In some embodiments, X is nitrogen.

In some embodiments, A is pyridine.

In some embodiments, the ligand-functionalized monolayer includes one or more ligands has the following formula:

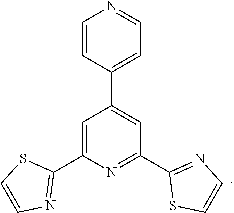 [SMILES: c1cc(-c2cc(-c3nccs3)nc(-c3nccs3)c2)ccn1]

In some embodiments, A is benzene.

In some embodiments, A-B is phenol.

In some embodiments, the ligand-functionalized monolayer includes one or more ligands having the following formula:

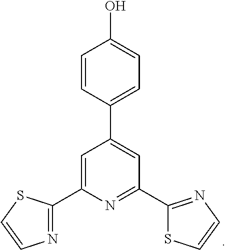 [SMILES: Oc1ccc(-c2cc(-c3nccs3)nc(-c3nccs3)c2)cc1]

In accordance with a broad aspect, a composite material for the detection of mercury. The composite material includes a ligand-functionalized monolayer; and a support material coupled to the ligand-functionalized monolayer. The ligand-functionalized monolayer includes one or more ligands having the formula:

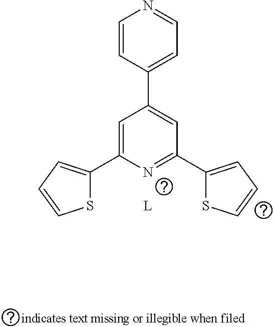

or the formula

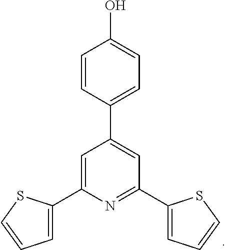 [SMILES: Oc1ccc(-c2cc(-c3cccs3)nc(-c3cccs3)c2)cc1]

In some embodiments, the composite material undergoes a fluorescence change in the presence of one or more target analytes.

In some embodiments, the fluorescence change is a quenching of fluorescence at a specific wavelength.

In some embodiments, the specific wavelength is between 300 nm and 600 nm.

In some embodiments, the target analytes include mercury.

In some embodiments, the ligand-functionalized monolayer includes TiO₂.

In some embodiments, the support material includes a nanoparticle.

In some embodiments, the nanoparticle is a Fe₃O₄ magnetic nanoparticle.

In accordance with a broad aspect, a method for the fluorescence detection of mercury is described herein. The method includes providing a fluorescence sensing indicator comprising the composite material of claim 1, exposing the indicator to a source of mercury and detecting any fluorescence changes.

In some embodiments, providing the fluorescence sensing indicator includes providing a fluorescence sensing indicator having a characteristic fluorescence wavelength and detecting any fluorescence changes includes detecting any fluorescence changes includes detecting a quenching of fluorescence at a characteristic wavelength.

In some embodiments, detecting a quenching of fluorescence at a characteristic wavelength includes detecting a quenching of fluorescence at a wavelength between 300 nm and 600 nm.

In accordance with a broad aspect, a method of removing mercury from a solution is described herein. The method includes providing the composite material of claim 1 to the solution containing mercury, the composite material being magnetic, and applying a magnetic field to the solution to remove the composite material and at least a portion of mercury from the solution.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A shows a plot showing ¹H NMR spectra and assignment of main protons of a ligand L (black) and L-Hg(II) complex (red).

FIG. 1B shows a plot showing a ¹H-¹H-COSY spectrum and assignment of main protons of L-Hg(II) complex.

FIG. 2A shows a front view and a bottom view of a schematic representation of the molecular structure of ligand L, wherein sulphur molecules are shown as being the largest molecules and nitrogen molecules are shown as being the smallest molecules. The remaining molecules are carbon.

FIG. 2B shows a front view and a bottom view of a schematic representation of the molecular structure of an L-Hg²⁺ complex in a trans orientation, where sulphur molecules are shown as being the second largest molecules, nitrogen molecules are shown as being the smallest molecules and a mercury molecule is shown as being the largest molecule. The remaining molecules are carbon.

FIG. 2C shows a front view and a bottom view of a schematic representation of the molecular structure of an L-Hg²⁺ complex in a cis orientation, where a sulphur molecule is shown in yellow, nitrogen molecules are shown in blue and mercury is shown in light shiny grey.

FIG. 2D shows a Jobs plot for formation of L-Hg²⁺.

FIG. 3A shows a plot of fluorescence emission intensity changes of a 4×10⁻⁵ M solution of ligand L in acetonitrile induced by addition of Hg²⁺ ions to the system, where excitation at 330 nm results in single excitation single emission turn off mercury detection by monitoring intensity at 413 nm.

FIG. 3B shows a plot of fluorescence emission intensity changes of 4×10⁻⁵ M solution of ligand L in acetonitrile induced by addition of Hg²⁺ ions to the system, where excitation at 385 nm results in single excitation double emission “turn off” at 413 and “turn on” at 563 nm detection of Hg²⁺ ions.

FIG. 4A is a schematic representation of anchoring ligand L onto a surface of a Fe₃O₄@TiO₂ nanoparticle.

FIG. 4B shows a representative SEM image of a Fe₃O₄@TiO₂-L composite material.

FIG. 4C shows a histogram of the size distribution for Fe₃O₄@TiO₂-L nanospheres.

FIG. 4D shows X-ray diffraction plots of Fe₃O₄ and Fe₃O₄@TiO₂.

FIGS. 4E and 4F show energy-dispersive X-ray spectroscopy (EDX) mapping of Fe₃O₄@TiO₂ nanoparticles.

FIG. 5A is a plot showing BET nitrogen adsorption-desorption isotherms of Fe₃O₄@TiO₂ (blue squares=adsorption, black squares=desorption).

FIG. 5B is a plot showing thermogravimetric analysis and differential thermal analysis of Fe₃O₄@TiO₂-L under argon.

FIG. 5C is a photograph of a magnetic Fe₃O₄@TiO₂-L nanocomposite colloidal solution in water before and after magnetic separation by an external magnetic field.

FIG. 5D is a fluorescence spectra of Fe₃O₄@TiO₂-L (1 mg of Fe₃O₄@TiO₂-L/3 mL of acetonitrile) after addition of different concentrations of Hg²⁺. The excitation wavelength was 330 nm. Arrow indicates the direction change in the fluorescence intensity.

FIG. 5E shows a plot of magnetic hysteresis curves of Fe₃O₄@TiO₂ and Fe₃O₄@TiO₂-L nanomaterials at 300K.

FIGS. 6A-6H are plots showing X-ray photoelectron spectra of Fe₃O₄@TiO₂-L (upper row A, C, E, G) and Fe₃O₄@TiO₂-L-Hg²⁺ (bottom row B, D, F, H) showing corresponding N 1s, Hg 4d, S 2s, and Si 2p/Hg 4f areas. Experimental data and curves of overall fitted spectra are shown. The Si 2p peak for the Fe₃O₄@TiO₂-L-Hg²⁺ has been deconvoluted: the peak at the middle (103.5 eV) represents the silicon from the silane template, while the peaks on the sides correspond to the Hg²⁺.

FIG. 7A shows an interference study with different metal ions with emission at 413 nm upon excitation at 330 nm in acetonitrile solution.

FIG. 7B shows an interference study with different metal ions with emission at 563 nm upon excitation at 385 nm in acetonitrile solution.

FIG. 7C shows a plot of CV response of the Fe₃O₄@TiO₂-L deposited on glassy carbon electrode and stepwise exposed to Hg²⁺ and Fe³⁺ in 0.1 M H₂SO₄ at a scan rate of 10 mVs⁻¹.

FIG. 8 shows a plot of differential pulse voltammetry (DPV) response of the Fe₃O₄@TiO₂-L deposited on glassy carbon electrode and stepwise exposed to Hg²⁺ and Fe³⁺ in 0.1M H₂SO₄.

FIG. 9A shows a plot of UV-visible spectra of the various metal salts coordinated to L₁.

FIG. 9B shows a plot of fluorescence emission spectra of the various metal salts coordinated to L₁.

FIG. 10A shows a plot of UV-visible spectra of the various metal salts coordinated to L₂.

FIG. 10B shows a plot of fluorescence emission spectra at an excitation wavelength of 337 nm of the various metal salts coordinated to L₂.

FIG. 11 shows plots of the change in fluorescence for Ligand L3 in the presence of different metals a) the fluorescence turn off emission peak at 383 nm. Following metal ions were checked. Al(III), As(III), Ba(II), Co(II), Cr(III), Cs(I), Cu(II), Fe(II), Fe(III), Hg(II), K(I), Li(I), Mg(II), Na(II), Pd(II), Ru(III), Sn(II), Zn(II).

Further aspects and features of the example embodiments described herein will appear from the following description taken together with the accompanying drawings.

### DESCRIPTION OF EXAMPLE EMBODIMENTS

Various apparatuses, methods and compositions are described below to provide an example of at least one embodiment of the claimed subject matter. No embodiment described below limits any claimed subject matter and any claimed subject matter may cover apparatuses and methods that differ from those described below. The claimed subject matter are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed subject matter. Any subject matter that is disclosed in an apparatus, method or composition described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should also be noted that, as used herein, the wording “and/or” is intended to represent an inclusive − or. That is, “X and/or Y” is intended to mean X or Y or both, for example. As a further example, “X, Y, and/or Z” is intended to mean X or Y or Z or any combination thereof.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

Recently, there has been a growing interest in the development of systems and methods for selectively detecting analytes of interest. Specifically, there has been growing interest in the development of composite materials for detecting mercury.

Composite materials that selectively absorb one or more analytes of interest are described herein. The composite materials are functionalized with one or more ligands that selectively absorb one or more analytes of interest. The composite materials generally exhibit a change in an optical property upon absorption of the one or more analyte of interest. The composite materials are generally suitable for applications such as, for example, an optical sensor for detecting one or more analytes in a medium. Methods of preparing such composite materials are also described herein.

As used herein, the term “absorbs” or “absorption” refers to the partitioning of an analyte into the composite material, or extraction of an analyte from a surrounding medium by the composite material. Such absorption may or may not be a reversible process. Such absorption is selective, in that non-analyte compounds present in the medium are not absorbed in any significant amount.

The composite materials described herein include a support material that is functionalized with one or more functionalizing ligands. The functionalizing ligand provides absorption of one or more analyte of interests. For instance, in accordance with at least one embodiment, the support material may be a porous support having a metal oxide surface comprising one or more chemical compounds such as, but not limited to, magnetite (Fe₃O₄), titanium oxide, silicon oxide, aluminum oxide, indium tin oxide, fluorine-doped indium tin oxide, iron oxide, zinc oxide; and natural complex metal oxides such as limestone (mostly calcium carbonate), diatomite (silica, alumina and iron oxide), and clay minerals (hydrous aluminum phyllosilicates), zeolites (aluminosilicates of sodium, potassium, calcium, and barium) or mixtures thereof, for example. In some specific embodiments, the support material may include magnetite and titanium oxide.

In accordance with at least one embodiment, composite materials for detecting mercury are described herein. The composite materials include a ligand-based (e.g. ligand-terminated) monolayer on a support material. Herein, the term ligand-terminated refers to a monolayer with one or more ligands forming an outermost point of the monolayer. In some embodiments, the support material has a metal oxide surface.

The ligand-based monolayer may be deposited on a surface of the support materials and is generally stable, uniform, and/or substantially free of contamination.

In at least one embodiment, the support material of the composite materials described herein is functionalized with at least one ligand. The at least one ligand may include bis(thienyl)-pyridine and/or bis(thiazole)-pyridine.

In some embodiments, the ligand is of formula (1), presented below:

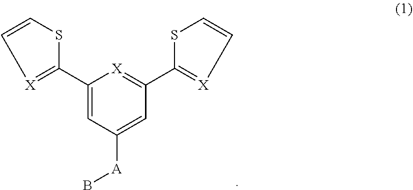

Hereinafter, the ligand of formula (1) is also referred to as ligand L.

In some embodiments, A is a linear or a cyclic aliphatic moiety, an aromatic ring or a fused aromatic ring system, a heteroaromatic ring, a fused heteroaromatic ring system, quaternary ammonium salt, or their combination, each of which may be optionally substituted.

In some embodiments, B is hydrogen, or a chemically derivatizable group, such as but not limited to alkene, alkyne, amino acid, azide, phosphate, phosphonate, carboxyl group, silane, siloxane, sulfate, quaternary ammonium salt, thiol, alkyl thiol, thioester or the like, for example, each of which may be optionally substituted.

In some embodiments, X is carbon or a heteroatom such as but not limited to nitrogen, sulphur or oxygen.

In some embodiments, the ligand is of formula (2), presented below:

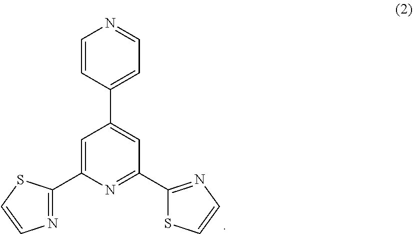 [SMILES: c1cc(-c2cc(-c3nccs3)nc(-c3nccs3)c2)ccn1]

Hereinafter, the ligand of formula (2) is also referred to as ligand L₁.

In some embodiments, the ligand is of formula (3), presented below:

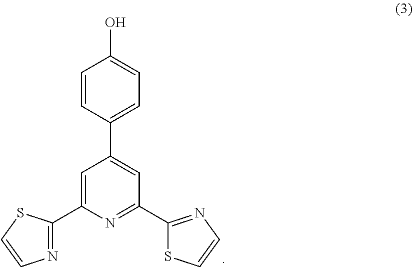 [SMILES: Oc1ccc(-c2cc(-c3nccs3)nc(-c3nccs3)c2)cc1]

Hereinafter, the ligand of formula (3) is also referred to as ligand L₂.

In some embodiments, the ligand is of formula (4), presented below:

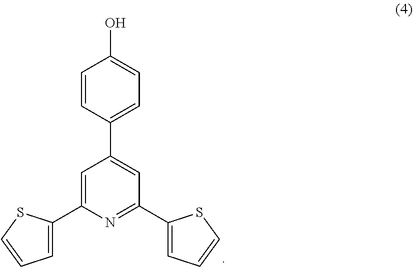 [SMILES: Oc1ccc(-c2cc(-c3cccs3)nc(-c3cccs3)c2)cc1]

Hereinafter, the ligand of formula (4) is also referred to as ligand L₃.

Anchoring (e.g. attaching) the ligands described herein, including but not limited to ligand L, onto appropriate support materials provides for the composite materials described herein to chemically adsorb analytes of interest such as, but not limited to, dissolved mercury, for example.

In some embodiments, absorption of the analyte of interest may be accompanied by a change in an optical property (e.g. fluorescence output change) of the resulting composite material. This change in an optical property can be used for identifying the presence of the analyte.

In some embodiments, dispersing of the composite material in aqueous or organic media provides for selective removal of an analyte of interest, such as but not limited to mercury, from the media.

In some embodiments, the support material is a magnetic core-shell nano-sphere that provides for the separation of mercury ions from the media.

In some embodiments, functionalization of conductive supports of indium tin oxide and fluorine-doped indium tin oxide nature provide for the fabrication of working electrodes for stripping voltammetry that allows determination of minor amounts of mercury via portable electrochemical methods.

In some embodiments, functionalization of conductive supports of indium tin oxide and fluorine-doped indium tin oxide nature provide for the fabrication of working electrodes for stripping voltammetry that allows determination of minor amounts of mercury via portable electrochemical methods.

In some embodiments, ligand L introduced above shows strong potential for mercury Hg(II) detection and uptake in solution. For example, upon excitation at 330 nm, ligand L performs as a single excitation (330 nm)-single emission (413 nm) selective “turn off” fluorimetric sensor for Hg²⁺ ions. The complex (e.g. ligand L and mercury) has a higher-energy excited state (e.g. E*=3.2-3.3 eV) with a high f (e.g. equal to about 0.40) for either conformer. The corresponding wavelength of ≈380 nm may more efficiently pump the complex.

In some embodiments, fluorimetric titration of ligand L by Hg(II) upon excitation at 385 nm, results in a concurrent decrease of the intensity of the emission band at 413 nm and the growth of the new emission peak at 564 nm. A well-defined isosbestic point (a specific wavelength, wavenumber or frequency at which the total absorbance of a sample does not change during a chemical reaction) at 498 nm suggests that no intermediates form during the event of uptake of mercury(II) ions by the ligand, L.

In some embodiments, binding between ligand L and Hg(II) occurs via SNS chelation and 1:1 stoichiometry between mercury and bis(thienyl)pyridine core of the ligand L. Equilibrium parameters geometry parameters for Hg²⁺-L were determined results suggest that two main conformers, cis and trans, are formed with very similar energy. For example, the cis conformer is calculated to be 0.07 eV higher in energy than the trans conformer, so both conformers are likely to co-exist. The potential energy barrier stabilizing the cis conformer is about 0.09 eV and corresponds to one S-containing ring being co-planar with the central ring.

In some embodiments, the resulting mercury coordination complex (L-Hg²⁺) can be isolated and fully characterized by ¹H ¹³C{¹H} NMR and HRMS to confirm the purity and the identity of the material.

In some embodiments, the ligand L is able to effectively detect mercury ions and differentiate Hg²⁺ from Zn²⁺, Cd²⁺, Cu²⁺, Cr³⁺, Co²⁺, Ru³⁺, and Fe²⁺ ions, for example, with minimum to no interference in solution (e.g. acetonitrile).

In some embodiments, the sensitivity of the detection was calculated for ligand L in acetonitrile solution. The limit of detection (LOD) for the “turn-off” peak of ligand L at 413 nm (λexc=380) is about 1.40 ppm of Hg²⁺.

In some embodiments, composite materials described herein (e.g. Fe₃O₄@TiO₂-L) for selective uptake with strong potential for Hg(II) uptake from aqueous and organic solutions were made by chemical anchoring of ligand L via siloxane chemistry on a surface-enhanced magnetite support.

In some embodiments, the composite materials described herein have a magnetic core to provide an easy mercury removal feature by applying an external magnetic field.

In some embodiments, the large size of the mercury ion is a limiting factor in the uptake properties. In some embodiments, about 36-37% of the ligand-based receptors on the support surface of the material form a coordination adduct with mercury ions. In some embodiments, upon full saturation by mercury ions, the composite materials described herein are able to uptake smaller Fe³⁺ ions.

In some embodiments, the composite materials described herein may be utilized as a single excitation (330 nm)-single emission (413 nm) sensor for Hg²⁺ ions.

In some embodiments, effective mercury uptake from aqueous solutions was studied by cold vapor atomic absorption that confirms mercury removal ability of the composite materials described herein as 13.35 μg of Hg²⁺ per one mg of the composite material.

In some embodiments, ligand L₁, introduced above, reacts with various analytes of interest, such as but not limited to Hg²⁺ and Fe²⁺. By the combination of UV-Vis and fluorimetery, mercury and iron(II) ions may be quantified and discriminated.

In some embodiments, ligand L₂, introduced above, reacts with various analytes of interest, such as but not limited to Fe²⁺ and Hg²⁺ detecting material. By the combination of UV-Vis and fluorimetery mercury and iron ions may be quantified and discriminated.

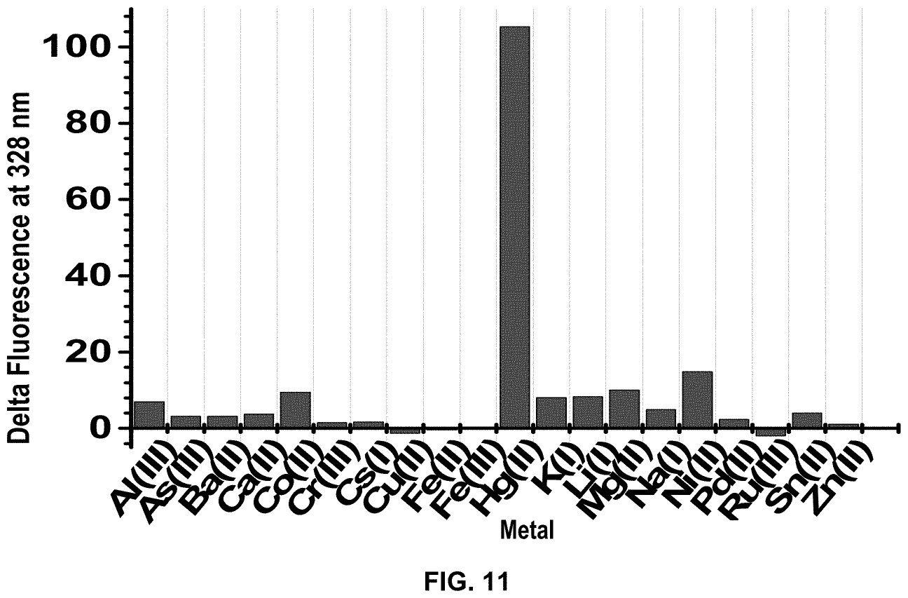

In some embodiments, ligand L₃, introduced above, reacts with various analytes of interest and can act as a selective “turn off” fluorescent sensor for mercury detection, due to its high affinity for mercury. Further, the binding stoichiometry from mercury to the ligand L₃ is about 1:1. No interference was detected by UV-Vis or fluorescence spectroscopy, in the presence of 17 other metals: (Al(III), As(III), Ba(II), Co(II), Cr(III), Cs(I), Cu(II), Fe(II), Fe(III), K(I), Li(I), Mg(II), Na(II), Pd(II), Ru(III), Sn(II), Zn(II)) to mercury (II) detection by ligand L₃ in mixture of water/acetonitrile solution. Unlike L₁ and L₂, no interference with Fe³⁺ was observed for the detection of Hg²⁺ (see FIG. 11).

In some embodiments, methods of removing mercury from a solution are described herein. The methods include providing a composite material described herein to the solution containing mercury. In these methods, the composite material is magnetic.

After a period of time, the composite material binds to at least a portion of the mercury in the solution. In some embodiments, mixing for the composite material and the solution may be required.

After the period of time, a magnetic field may be applied to the solution to remove the composite material and at least a portion of mercury bound to the composite material from the solution.

To get a better understanding of the subject matter described herein, the following working examples are set forth. It should be mentioned that these examples are only for illustrative purposes and they are not limiting the scope of the claimed subject matter in any way.

### Examples

### Synthesis of the Ligand 2,6-di(thiophen-2-yl)-4,4′-bipyridine (L)

Ligand L was synthesized according to previously published procedures (see for example Constable, E. C.; Thompson, A. M. J. Chem. Soc. Dalton Trans. 1992, (20), 2947-2950; Thapa, P.; Karki, R.; Basnet, A.; Thapa, U.; Choi, H.; Na, Y.; Jahng, Y.; Lee, C.-S.; Kwon, Y.; Jeong, B.-S.; Lee, E.-S. Bull. Korean Chem. Soc. 2008, 29 (8), 1605-1608).

Ligand L was shown to have the following properties: ¹H-NMR (400 MHz, DMSO-d₆) δ 8.79 (dd, J=4.4, 1.7 Hz, 2H), 8.21 (s, 2H), 8.07 (dd, J=3.7, 1.1 Hz, 2H), 8.04 (dd, J=4.5, 1.7 Hz, 2H), 7.71 (dd, J=5.0, 1.1 Hz, 2H), 7.23 (dd, J=5.0, 3.7 Hz, 2H). ¹³C NMR (101 MHz, DMSO-d₆) δ 152.99 (s), 150.91 (s), 147.12 (s), 144.68 (s), 144.31 (s), 129.58 (s), 128.94 (s), 126.85 (s), 114.85 (s), FT-IR: v/cm⁻¹ 3044w (C—H aromatic), 2100w (C—H aromatic), 1535m (C═S), 1455s (C═C—C), 1066m (C—H aromatic), 817vs (C—H aromatic), 691vs (C—H aromatic). ESI-MS: For C₁₈H₁₂N₂S₂ predicted 320.44, found (M+1) 321.05.

### Synthesis of the Ligand 1-methyl-2′,6′-di(thiophen-2-yl)-[4,4′-bipyridin]-1-ium (Hereinafter Referred to as QL, which has the Following Structure)

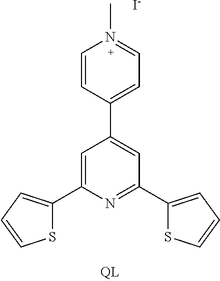

Following a literature procedure [Goodall, W.; Williams, J. A. G., J. Chem. Soc., Dalton Trans. 2000, (17), 2893-2895] a reflux system was assembled whilst hot and flushed with N_{2(g)}. To the round bottom flask, ligand L (0.16 mmol), acetonitrile (25 mL) and methyl iodide (0.78 mmol) were added then heated to 40° C. whilst stirring. Upon reaching 40° C. the reaction mixture was refluxed for 24 hours. Once cooled to room temperature, the solvent was removed by a rotary evaporator and the powder dried in vacuo to give 1-methyl-2′,6′-di(thiophen-2-yl)-[4,4′-bipyridin]-1-ium as a bright yellow solid, QL (35 mg, 67%).

Ligand QL was found to have the following properties: ¹H-NMR (400 MHz, DMSO-d₆) δ 9.19 (d, J=6.8 Hz, 2H), 8.81 (d, J=6.8 Hz, 2H), 8.40 (s, 2H), 8.09 (dd, J=3.7, 1.0 Hz, 2H), 7.74 (dd, J=5.0, 1.0 Hz, 2H), 7.25 (dd, J=5.0, 3.7 Hz, 2H) 4.39 (s, 3H). ¹³C NMR (101 MHz, DMSO-d₆) δ 148.65 (m), 146.94 (s), 141.4 (s), 138.73 (s), 138.27 (s), 125.08 (s), 123.94 (s), 122.29 (s), 120.45 (s), 110.15 (s), 42.93 (s) FT-IR: v/cm⁻¹ 2991w (C—H aromatic), 2100w (C—H aromatic), 1539m (C═S), 1419s (C═C—C), 830s (C—H aromatic), 709vs (C—H aromatic).

### Synthesis of L-Hg²⁺ Metal Complex (Structure Shown Below)

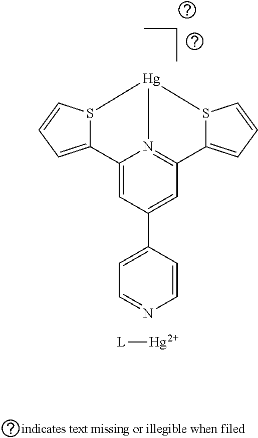

Corresponding mercury complex L-Hg²⁺ was formed when a solution of 30.2 mg (0.076 mmol) of mercury(II) perchlorate hydrate in acetonitrile (2 mL) was added to a solution of L (24.2 mg, 0.076 mmol) in acetonitrile (3 mL). After 30 min yellow precipitate was filtered out and washed with 50 mL of hexanes resulting in 10 mg, 25.4% yield of complex L-Hg²⁺.

For L-Hg²⁺, the following properties were observed: ¹H NMR: (400.00 MHz, CD₃CN): δ 8.88 (d, ³J_{HH}=6.1 Hz, 1H), 8.49 (d, ³J_{HH}=6.2 Hz, 1H), 8.07 (s, 1H), 7.92 (d, ³J_{HH}=3.6 Hz, 1H), 7.65 (d, ³J_{HH}=5.0 Hz, 1H), 7.26 (m, 1H). ¹³C NMR (101 MHz, CD₃CN): δ 155.99 (C_{q}), 153.12 (C_{q}), 142.53, 130.47, 129.13, 127.70, 126.26, 126.22 (C_{q}), 116.5. Assignments for quaternary carbons were made by comparison of ¹³C NMR to DEPT 135-NMR. ESI-MS: For C₁₈H₁₂ HgN₂S₂²⁺ predicted 261.00, found (M−1) 260.11, (M−3) 258.04, (M−3+K) 283.05.

### Synthesis of QL-Hg²⁺ Metal Complex (Structure Shown Below)

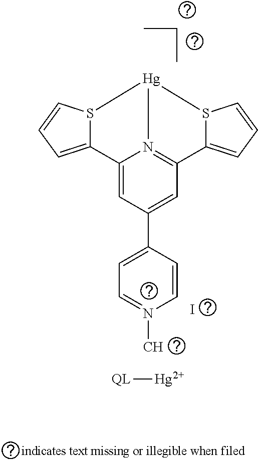

The QL-Hg²⁺ complex was synthesized by addition to the solution of mercury(II) perchlorate hydrate (23.4 mg, 0.058 mmol) in acetonitrile (2 mL) to a solution of ligand L (27.0 mg, 0.058 mmol) in acetonitrile (3 mL) After 30 min, the yellow precipitate was filtered out and washed with 50 mL of hexanes resulting in 7.6 mg, 17.8% yield of complex QL-Hg²⁺. ¹H NMR (400 MHz, CD₃CN) δ 8.81 Hz (d, ³J_{HH}=7.24 Hz, 1H) 8.46 Hz (d, ³J_{HH}=7.24 Hz, 1H) 8.09 Hz (s, 1H) 7.94 Hz (d, ³J_{HH}=4.84 Hz, 1H) 7.71 Hz (d, ³J_{HH}=6.04 Hz, 1H) 7.28 Hz (m, 1H) 4.39 (s, 3H). ¹³C-NMR (101 MHz, CD₃CN) δ: 153.1 (C_{q}), 152.6 (C_{q}), 145.9, 144.7 (C_{q}), 137.7 (C_{q}).

A solution was created with 0.95 ml of 4-pyridine carboxaldehyde in 85 ml of ethanol, in a 500 ml rb flask. Before adding the solution to the rb flask, a portion of the ethanol was taken out to dissolve 1.56 grams of KOH pellets in a beaker. Afterwards, 2.2 ml of Acetylthiazole was added to the solution. The KOH solution that was prepared earlier was added dropwise. After five minutes, 35 ml of NH₄OH was added at a quick rate with a pipette and left to stir for 3 days. The white precipitate was formed and filtered out with a Hirsh funnel with 3 ethanol washes. Some filtrate that had passed through initially was collected however it maintained the orange color instead of the white. The result was a Product Yield: 79%. The NMR was also consistent with previously published material.

### Synthesis of -(2,6-di(thiazol-2-yl) pyridin-4-yl) phenol (L₂)

This synthesis was performed following a previously reported procedure (see Durrell, A., Li, G., Koepf, M., Young, K., Negre, C., & Allen, L. et al. 2014 Journal of Catalysis, 310, 37-44. doi: 10.1016/j.jcat.2013.07.001), with minor modifications. The synthesis of L₂ began by dissolving 4-hydroxybenzaldehyde (1.24 g, 10.16 mmol) in water (5 mL), followed by the addition of NaOH (1.48 g, 37.1 mmol) in EtOH (10 mL). 2-acetylthiazole (2.20 mL, 21.07 mmol) was added to the solution, in which the mixture turned a deep dark red, and was stirred for 1 hr. NH₄OH (50 mL, xx mM) was added to the reaction and stirred at room temperature for 24 hrs. The product was obtained via a suction filtration, and was washed with DI water and EtOH. The product was a white/yellow precipitate and yielded: 3.599 g (55%). ¹H NMR (400 MHz, DMSO) δ ppm 9.83 (s, OH, 1) 8.21 (s, 2H, 4) 8.02 (d, J_{HH}=0.99, 2H, 5) 7.88, 7.87 (d, J_{HH}=0.94, 2H, 6) 7.50, 7.48 (d, J_{HH}=1.02, 2H, 3) 6.30, 6.28 (d, J_{HH}=1.02, 2H, 2)

### Synthesis of the 4-(2,6-di(thiophen-2-yl)pyridine-4-yl)phenol, L₃

In a 50 mL round bottom flask, 10 mmol of 4-hydroxybenzaldehyde was added to 20 mmol of 2-acetylthiophene along with 10 mL of ethanol and 5 mL of deionized water and 26 mmol of sodium hydroxide. The reaction mixture was stirred for 1 hour at room temperature. The first time this synthesis was performed 30 mL of ammonium hydroxide was added to the round bottom flask and stirred for 24 hours. No precipitate formed as predicted so another 15 mL of ammonium hydroxide was added with still no precipitate formed. A liquid-liquid extraction was done with dichloromethane and the organic layer (20 mL×3 times), was collected and all volatiles were removed to produce a crude brown oil. The aqueous layer was extracted with ethyl acetate (15 mL×3 times), to attempt to collect more product. Solvent was evaporated in vacuo. An excess of ammonium hydroxide was added to the residue along with 10 mL of ethanol to wash the product of impurities. This reaction was allowed to stir for 48 hours at room temperature. After the 48 hours a white precipitate formed and was collected through suction filtration. The ¹H-NMR of this product (L3). ¹H-NMR (400 MHz, DMSO-d₆) δ 9.108 ppm (s, 2H), 7.935 ppm (td, 4H, J=5.3, 3.1 Hz), 7.201 ppm (t, 2H, J=8.2 Hz), 7.072 ppm (d, 2H, J=6.7 Hz), 6.574 ppm (d, 2H, J=7.8 Hz) IR (cm⁻¹): 3200, 3106, 3080, 1615, 1613, 1522, 1360, 1227, 1200, and 750.

This synthesis was performed a second time to achieve a higher yield. It followed the same procedure, however, a larger excess of ammonium hydroxide (50 mL) was added to ensure the product was fully converted from the intermediate. In addition, the extraction was done fully with EtOAc as that was shown to increase the amount of product in the organic layer. Isolated yield of the final product (L) was 0.6587 g (19.3%).

### Synthesis of the Fe₃O₄ Nanoparticles

The synthesis was carried out according to a previously reported method with modification, (see Ma, W.-F.; Zhang, Y.; Li, L.-L.; You, L.-J.; Zhang, P.; Zhang, Y.-T.; Li, J.-M.; Yu, M.; Guo, J.; Lu, H.-J.; Wang, C.-C., ACS Nano 2012, 6 (4), 3179-3188; Deng, H.; Li, X.; Peng, Q.; Wang, X.; Chen, J.; Li, Y., Angew. Chem. 2005, 117 (18), 2842-2845).

2.5 g of FeCl₃.6H₂O was allowed to stir in 75 mL of ethylene glycol until it dissolved. Then 7.2 g of sodium acetate and 2 g of polyethylene glycol (PEG) 4000 were added to the above solution and stirred until all the reactants dissolved. The mixture was then transferred into a Teflon-lined stainless steel autoclave. The autoclave was heated to and maintained at 160° C. for 8 hours and then naturally cooled to room temperature. The product mixture was centrifuged, the liquid was discarded while the solids were washed with ethanol and water. The magnetite product was dried under vacuum at 90° C. for 10 hours.

### Synthesis of the Fe₃O₄@TiO₂ Nanoparticles

Based on a pervious method, [Yu, J.; Su, Y.; Cheng, B.; Zhou, M.; J. Mol. Catal. A: Chem. 2006, 258 (1-2), 104-112], 100 mg of Fe₃O₄ microspheres were dispersed in 100 mL of an ethanol/acetonitrile (3/1, v/v), followed by the addition of 1 mL concentrated (28%) ammonia solution under sonication for 20 minutes. Afterwards 1.6 mL of tetrabutyl titanate (TBOT) in 30 mL of ethanol/acetonitrile (3/1, v/v) was added dropwise under continuous sonication. The mixture was then allowed to stir under sonication for 2 hours then transferred into a Teflon-lined stainless steel autoclave. The autoclave was heated to and maintained at 160° C. for 24 hours and then naturally cooled to room temperature. The product mixture was centrifuged, the liquid was discarded while the solids were washed with ethanol and water. The product was then dried under vacuum at 100° C. overnight. The powder was sonicated in solutions of ethanol and water multiple times then separated with a magnet to remove any unreacted TiO₂.

### Synthesis of Fe₃O₄@TiO₂-L Nanoparticles

The solid substrate Fe₃O₄@TiO₂ NP was functionalized by the molecular receptor ligand L by a two step procedure using chlorobenzylsiloxane-based templating layer according to an adapted literature procedure (see Choudhury, J.; Kaminker, R.; Motiei, L.; Ruiter, G. d.; Morozov, M.; Lupo, F.; Gulino, A.; Boom, M. E. v. d., Linear vs Exponential Formation of Molecular-Based Assemblies. J. Am. Chem. Soc. 2010, 132 (27), 9295-9297).

Under N₂ atmosphere, Fe₃O₄@TiO₂ NP substrate was submerged into a solution of trichloro(4-(chloromethyl)phenyl)silane with anhydrous hexane (1:200 v/v) for 20 min. The material was washed 3× with anhydrous hexane then with anhydrous acetonitrile, and sonicated 1× for 5 min per solvent. Then the material was submerged into the solution of ligand L (0.2 mM) in anhydrous acetonitrile and sealed in a pressure tube. The material was heated for 96 h at 95° C. without light. After cooling down, the resulting Fe₃O₄@TiO₂-L nanoparticle material was washed 3× with anhydrous hexane then anhydrous acetonitrile, and sonicated 1× for 5 min per solvent.

### Determining Selectivity of Ligand L to Various Metal Ions in Acetonitrile

A stock solution of L was made in acetonitrile to give a final concentration of 9.98×10⁻³ mM. Eight metal (Fe²⁺, Fe³⁺, Cr³⁺, Zn²⁺, Co²⁺, Ru³⁺, Cd²⁺, Cu²⁺) solutions were prepared by dissolving the corresponding metal salt in acetonitrile. An aliquot of the ligand L stock solution (9.98×10⁻³ mM) was transferred to a 10 mm×10 mm quartz cuvette. The fluorescence emission was measured using λₑₓ=330 nm and λₑₘ=340-640 nm. An aliquot of the first metal solution M was added to the cuvette, stirred for 2 minutes, then the fluorescence emission of M+L was measured. Hg²⁺ was then added to the cuvette, stirred for 2 minutes before the fluorescence emission of M+L+Hg²⁺ was obtained. These steps were repeated for all above eight metal salts.

### Fluorescence Emission Experiment of L-Hg²⁺ Complex Formation

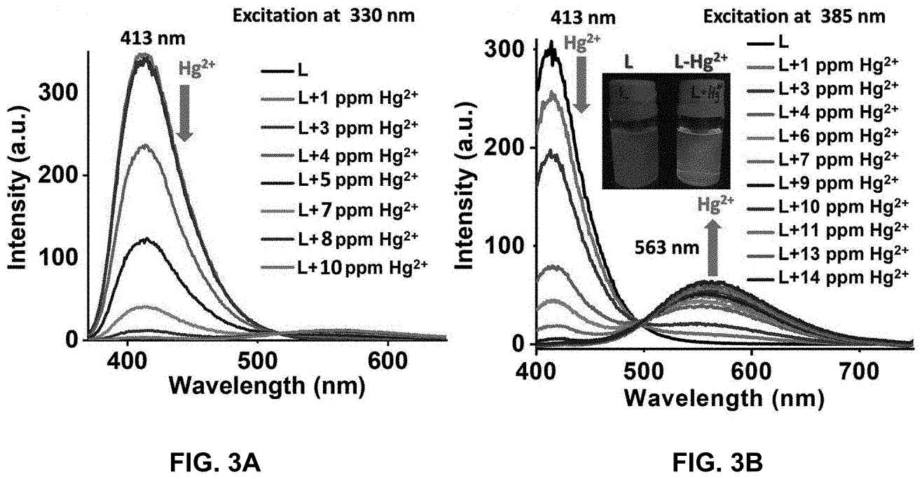

A stock solution of Hg²⁺ was prepared by dissolving Hg(ClO₄)₂ in acetonitrile. An aliquot of the L stock solution (1×10⁻⁴ mM) was transferred to a 10 mm×10 mm quartz cuvette. Additions of Hg²⁺ were added via a microsyringe to the L aliquot solution until the fluorescence peak at 413 nm was fully quenched (see FIG. 3A). Between each addition step, the solution was mixed for 30 sec before the fluorescence emission was measured. The experiment was performed two times under excitation wavelengths of 325 and 385 nm, respectively. When the sample was exited under 385 nm upon addition of mercury, in addition to the disappearance of the peak at 413 nm, the growing of the new emission peak at 580 nm was observed (see FIG. 3B).

### Determination of Fluorescence Quantum Yields for L and L-Hg²⁺

### Quantum Yield of Ligand L at 413 nm

The fluorescent standard sample to be used was L-tryptophan as its λ_{abs} and λₑₘ are similar to that of the ligand L test sample. A stock solution of L-tryptophan was prepared by dissolving L-tryptophan (20 mg) in DI water to give a concentration of 10 mM. This was followed by two further dilutions of the solution to give a final concentration of 0.2 mM. The fluorescence emission was measured using λₑₓ=280 nm and λₑₘ=290-500 nm. This was repeated for the L test sample, where the solvent background used was acetonitrile and the concentrations of the five dilutions were 1.11×10⁻³ mM, 2.22×10⁻³ mM, 3.33×10⁻³ mM, 4.44×10⁻³ mM and 5.55×10⁻³ mM. Fluorescence emission was measured using λₑₓ=330 nm and λₑₘ=340-550 nm. The integrated fluorescence intensity was plotted against the absorbance at the fluorometer excitation wavelength. This is at 280 nm for L-tryptophan and 330 nm for L. A linear regression line was fitted to the resulting graph, of which the gradient is required for the quantum yield calculation.

Equation 1 (see Williams, A. T. R.; Winfield, S. A.; Miller, J. N., Analyst 1983, 108 (1290), 1067-1071) is required to calculate the fluorescence quantum yield:

$${φ}_{x} = {φ}_{STD} ( \frac{{m}_{x}}{{m}_{STD}} ) ( \frac{{η}_{x}^{2}}{{η}_{STD}^{2}} ) \qquad ( 1 )$$

where ‘x’ denotes the complex (test sample) and ‘STD’ denotes L-tryptophan (standard sample). φ represents the quantum yield, m represents the gradient of the plot of integrated fluorescence intensity vs absorbance, and η represents the refractive index of the solvent used.

### Propagation of Error for Quantum Yield Calculations

Equation 2 was used to calculate the standard deviation from quantum yield.

$${σ}_{x} = {φ}_{x} \sqrt{{( \frac{{σ}_{m x}}{{m}_{x}} )}^{2} + {( \frac{{σ}_{m S T D}}{{m}_{S T D}} )}^{2} + {( \frac{{σ}_{φ STD}}{{φ}_{STD}} )}^{2}} \qquad ( 2 )$$

The standard deviation from quantum yield for ligand L was calculated using equation 2.

$${σ}_{x} = 0.21 \sqrt{{( \frac{2.14 \times {10}^{4}}{2.88 \times {10}^{5}} )}^{2} + {( \frac{4.55 \times {10}^{4}}{1.59 \times {10}^{5}} )}^{2} + {( \frac{0.01}{0.12} )}^{2}} = 0.08$$

Variables for calculation of the standard deviation from quantum yield for L are shown in Table 1.

TABLE 1
Variables for calculation of the standard
deviation from quantum yield for L

| Parameter | Value | Standard Error (±) |
|---|---|---|
| Quantum Yield, ϕₓ, L | 0.21 | 0.08 |
| mₓ | 2.88 × 10⁵ | 1.07 × 10⁴ |
| m_{STD} | 1.59 × 10⁵ | 2.27 × 10⁴ |
| ϕ_{STD} | 0.12 | 0.01 |
| σₘₓ = standard error of mₓ * \sqrt{N} | 2.14 × 10⁴ | — |
| σ_{mxSTD} = standard error of m_{STD} * \sqrt{N} | 4.55 × 10⁴ | — |

### Quantum Yield of L-Hg²⁺ Complex at 585 nm

The quantum yield of L-Hg²⁺ was determined using the fluorescent standard sample Ru(bipy)₃ as its λ_{abs} and λₑₘ are similar to that of the L-Hg²⁺ complex test sample. (see Rurack, K., Fluorescence Quantum Yields: Methods of Determination and Standards. In Standardization and Quality Assurance in Fluorescence Measurements I: Techniques, Resch-Genger, U., Ed. Springer Berlin Heidelberg: Berlin, Heidelberg, 2008; pp 101-145). A stock solution of Ru(bipy)₃ was prepared by dissolving Ru(bipy)₃ (2.5 mg) in DI water (20 mL) to give a concentration of 2.0*10⁻⁴ M. A stock solution of L-Hg²⁺ was prepared by dissolving L (42.8 mg) and Hg(ClO₄)₂ (31.6 mg) in acetonitrile (3 mL), the L-Hg²⁺ (4.1 mg) was then filtered out and dissolved in acetonitrile (10 mL), to give a final concentration of 6.7*10⁻⁴ M solution.

The UV-Vis absorbance of the solvent background was measured, followed by eleven dilutions of the standard Ru(bipy)₃ stock solution. The fluorescence emission was also measured using λₑₓ=452 nm and λₑₘ=460-700 nm. This was repeated for the L-Hg²⁺ test sample, fluorescence emission was measured using λₑₓ=380 nm and λₑₘ=400-700 nm. The integrated fluorescence intensity was plotted against the absorbance at the fluorometer excitation wavelength. This is at 452 nm for Ru(bipy)₃ and 380 nm for L-Hg²⁺. A linear regression line was fitted to the resulting graph, of which the gradient is required for the quantum yield calculation.

Equation 1, above, was used to calculate the fluorescence quantum yield, where ‘x’ denotes the complex L-Hg²⁺ (test sample), ‘STD’ denotes Ru(bipy)₃ (standard sample), φ represents the quantum yield, m represents the gradient of the plot of integrated fluorescence intensity vs absorbance, and η represents the refractive index of the solvent used.

φₓ=0.57

Propagation of error for quantum yield calculations for L-Hg²⁺-Complex Equation 2, above, was used to calculate the standard deviation from quantum yield for L-Hg²⁺ complex.

$${σ}_{x} = 0.57 \sqrt{{( \frac{6.48 \times {10}^{3}}{3.24 \times {10}^{4}} )}^{2} + {( \frac{4.14 \times {10}^{3}}{2.03 \times {10}^{4}} )}^{2} + {( \frac{0.01}{0.36} )}^{2}} = 0.16$$

Fluorescence Emission Experiment of Hg²⁺ with Fe₃O₄@TiO₂-L Nanoparticles

The fluorescence samples of Fe₃O₄@TiO₂-L NP were prepared by adding 1.0 mg of Fe₃O₄@TiO₂-L nanoparticles into 3.0 mL of anhydrous acetonitrile and sonicating it for 15 minutes. The solution was then transferred to a 10 mm×10 mm quartz cuvette. The fluorescence emission was measured using λₑₓ=330 nm and λₑₘ=340-600 nm at a slow scan rate. A stock solution of Hg²⁺ was prepared by dissolving Hg(ClO₄)₂ in acetonitrile, which was added drop wise to the solution in intervals of 1.0 μL using a microsyringe. The fluorescence spectra of the Fe₃O₄@TiO₂-L NP with Hg²⁺ were measured in triplicates to obtain the average peak height. Between each run and addition, the solution was mixed for 30 sec before the fluorescence emission was measured.

Selectivity Experiment of Hg²⁺ with Fe₃O₄@TiO₂-L Nanoparticles

Eight metal solutions (Fe²⁺, Fe³⁺, Cr³⁺, Zn²⁺, Co²⁺, Ru³⁺, Cd²⁺, and Cu²⁺) were prepared by dissolving the corresponding metal salt in acetonitrile. 1.0 mg of Fe₃O₄@TiO₂-L NP was added into 3.0 mL of anhydrous acetonitrile and sonicated for 15 minutes then transferred to a 10 mm×10 mm quartz cuvette. The fluorescence emission was measured using λₑₓ=330 nm and λₑₘ=340-640 nm. An aliquot of the first metal solution was added to the cuvette, sonicated for 2 minutes. Then the fluorescence emission of M+L was measured. Hg²⁺ was then added to the cuvette, sonicated for 2 minutes before the UV-vis and fluorescence emission of M+L+Hg²⁺ was obtained. These steps were repeated for all the above eight metal salts.

Mercury Uptake Experiment by Fe₃O₄@TiO₂-L Nanoparticles from Aqueous Solutions

A Cold Vapour atomic absorption (AA) method was employed to study mercury uptake ability for Fe₃O₄@TiO₂-L NP nanomaterial as previously reported (see Xiang, G.; Li, L.; Jiang, X.; He, L.; Fan, L., Anal. Lett. 2013, 46 (4), 706-716). In this experiment, a mass of 62.0 mg of mercury perchlorate was weighed out, and then dissolved in 100 mL of type 1 DI water in a 100 mL volumetric flask to create an initial stock solution of 274 mg/L mercury. Calibration solutions and test solutions were prepared by stepwise dilution of the stock solution. Mercury uptake ability was determined in triplicates to ensure reliable measures of the uptake properties. For the mercury uptake experiment, samples were created by adding 7.5 mL of the working 1 mg/L stock solution to the 30 mL sample vials. Then 1.7-1.9 mg of Fe₃O₄@TiO₂-L NP were added to the vial and sonicated for 15 minutes to allow for complete exposure to the solution. Following this, the reacted Fe₃O₄@TiO₂-L NP material was removed from the media using a magnet, the solutions were quantitatively transferred to a 100 mL volumetric flask and diluted to 100 mL using DI water. Mercury content was measured using a Varian AAS 240 instrument equipped with a cold-vapour absorption set-up, using stannous chloride as the reductant.

Mercury absorption ability of the Fe₃O₄@TiO₂-L nanoparticles was determined as 13.35 μg Hg²⁺/mg of material.

Table 2 shows results of a Mercury uptake analysis by Fe₃O₄@TiO₂-L NP material.

TABLE 2
Mercury uptake analysis by Fe₃O₄@TiO₂-L NP material.
Mercury Uptake Analysis

| | Concentration | | | Concentration | Mercury | | |
|---|---|---|---|---|---|---|---|
| | Hg | Mass of | | After | Uptake/ | | |
| | Initially | Magnetite | | Addition and | mg of | Mercury | Mercury |
| | Added | NP's | Absorbtion | Removal of | NP's | Uptake | Uptake |
| Sample | (μg/L) | (mg) | Measured | NP's (μg/L) | (μg/L) | (mg/mg) | (μg /mg) |
| 1 | 75 | 1.7 | 0.3323 | 50.79 | 14.24 | 0.01424 | 14.24 |
| 2 | 75 | 1.9 | 0.3312 | 50.65 | 12.81 | 0.01281 | 12.81 |
| 3 | 75 | 1.8 | 0.3386 | 51.59 | 13.01 | 0.01301 | 13.03 |
| Average | 75 | 1.8 | 0.3340 | 51.01 | 13.35 | 0.01335 | 13.35 |

### Binding Constants Calculations

A modified Stern Volmer equation, as in Equation 3 shown below, was used to calculate the binding constants:

$$log \frac{{F}_{0} - F}{F} = {log K}_{b} + n log [ Q ] \qquad ( 3 )$$

where F₀ is the fluorescence intensity of L at 413, F is the intensity of L at 413 nm in the presence of Hg²⁺, Kb is the binding constant, n is the number of binding sites (n=1 for our system) and [Q] is the concentration of Hg²⁺.

### Limit of Detection Calculations

The limit of detection (LOD) was calculated from the calibration curves using the following Equation 4 where σ is the standard deviation of the response.

$$L O D = \frac{3 σ}{slope} \qquad ( 4 )$$

### Electrochemistry

An ink was made by sonicating 2.7 mg of the Fe₃O₄@TiO₂-L nanoparticles, 100 μL DI water, 100 μL isopropyl alcohol, and 50 μL Nafion®. 2 μL of the ink was drop coated onto a 0.071 cm² diameter glassy carbon electrode and dried with heat (loading of the material: 304 μg/cm²). The functionalized electrode was immersed into a 0.6 mM solution of Hg²⁺ for 30 min. The electrode was washed and corresponding electrochemical tests were ran. The electrode was then immersed in a 5 mM solution of Fe³⁺ for 30 min. The electrode again was washed with water and electrochemical tests were performed.

Electrochemical measurements were run in 0.1M H₂SO₄. A mercury/mercury sulfate was used as a reference electrode and a platinum wire was used as the counter electrode. Cyclic voltammetry (CV) was performed at 50 mV/s and 10 mV/s in the potential range of 0-1.2V vs SHE. The electrochemical measurements were performed using a Solartron Analytical 1470E potentiostat with corresponding Multistat and CView software. Differential pulse voltammetry was run with a height of 50 mV, a width of 10 ms, a period of 100 ms, and an increment of 10 mV on a Pine wavedriver with corresponding aftermath software.

### Exploring L-Hg(II) Complex Formation

The ligand-to-metal coordination mode is an important parameter that determines the efficiency of metal uptake. It is documented in the literature that hydrogen in ortho-position of thiophene could be replaced to form derivatives and to be involved in coordination with transition metals (see for example A. K. Shigemoto, C. N. Virca, S. J. Underwood, L. R. Shetterly and T. M. McCormick, J. Coord. Chem., 2016, 69, 2081-2089). Thus, structurally related 2,6-bis(2-thienyl)pyridine-based molecular receptor was reported to coordinate with mercury through cyclometalation via one carbon of thiophene by CNS chelating mode (A. K. Shigemoto, C. N. Virca, S. J. Underwood, L. R. Shetterly and T. M. McCormick, J. Coord. Chem., 2016, 69, 2081-2089).

In order to unambiguously determine the coordination mode of the ligand L to the Hg(II), comprehensive NMR, DFT studies, and chemical titration (Jobs plot) were performed. The results of experimental and theoretical characterization of the system are fully consistent with the formation of 1:1 L:Hg SNS type of chelate.

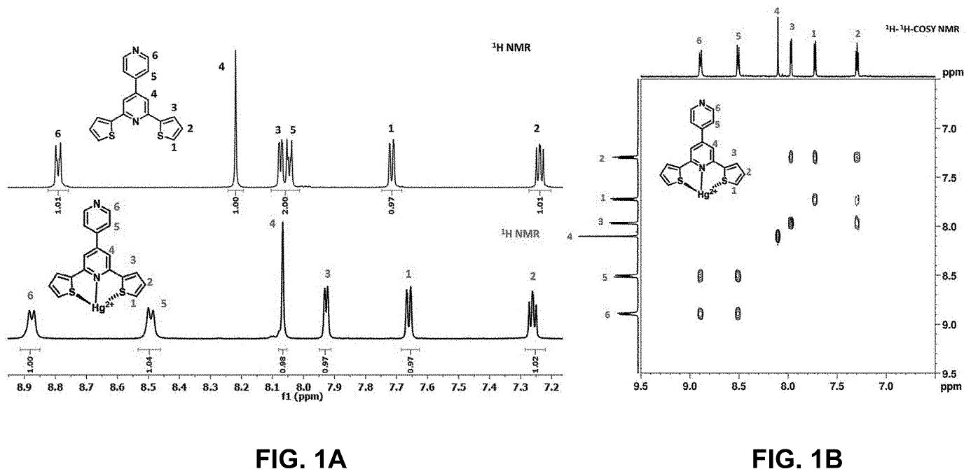

¹H NMR spectrometry (see FIGS. 1A and 1B) demonstrates that there is no evidence of hydrogen abstraction that would be consistent with the formation of the cyclometalated CNS-complex. Moreover, no breaking of the symmetry was observed upon coordination of L to the Hg(II), as this would be expected for asymmetric CNS coordination mode.

Significant downfield shifts for characteristic doublets corresponding to non-chelating pyridine ring from 8.78 and 8.04 ppm (in the free L) to 8.90 and 8.49 ppm (in the complex), respectively, were observed. In addition, singlet resonance at 8.22 ppm of the protons of chelating pyridine ring (4) and doublet resonance at 8.07 ppm for the thiophene protons (3) become noticeably shifted upfield to 8.07 ppm and 7.93 ppm, respectively. Shifts of two other thiophene protons (1 and 2) upon chelating Hg(II) are less distinct. No other products/intermediates were detectable by ¹H-NMR.

¹H-NMR observations are fully consistent with the SNS coordination mode when both sulfur atoms of thiophene rings and the nitrogen atom of the middle pyridine unit form a symmetrical structure.

### DFT Studies and Jobs Plot

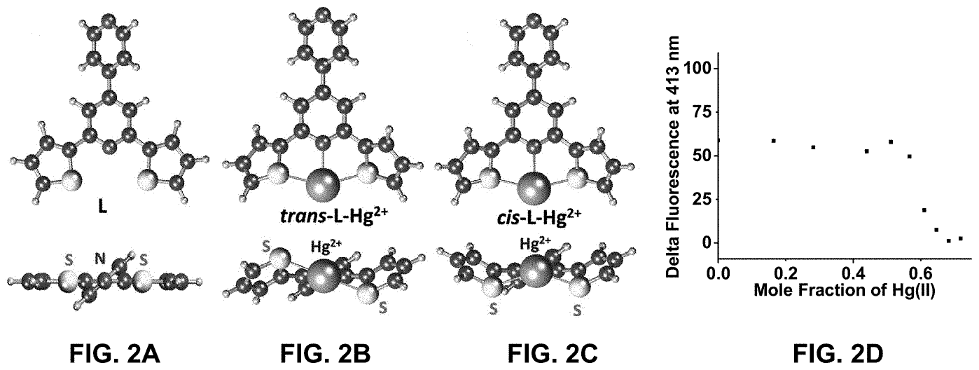

The molecular structures of L (see FIG. 2) and L-Hg²⁺ were established on the basis of Density Functional Theory (DFT) studies (see Table 3).

TABLE 3
Equilibrium geometry parameters for Hg²⁺⁻L
determined by DFT (Density Functional Studies)

| Conformer | r(Hg − N)/Å | r(Hg − S)/Å | θ(S − Hg − N − S)/° |
|---|---|---|---|
| cis | 2.33 | 2.69 | 141 |
| trans | 2.34 | 2.70 | 180 |

The DFT studies confirm the SNS coordination mode with two very similar in energy cis and trans geometries around the mercury ion. In addition, the DFT results allowed for ruling out the CNS binding mode as a coordination adduct with a considerably higher energy.

To study in depth the geometry of ligand L and relative stability of possible conformers of L-Hg²⁺, DFT calculations were performed. The optimized free ligand has co-planar central and S-carrying rings, and the outer ring twisted at 39° to this plane (see FIG. 2A). The S—S distance is about 4.48 Å. In the L-Hg²⁺ complex, such S—S separation appears to be too small to accommodate the mercury dication, so the S-carrying rings twist appropriately as well, moving the S atoms further apart.

Two conformers arise here, labeled cis and trans (see FIGS. 2B and 2C)—with the S atoms shifted in the same or opposite directions, so that they are on the same or opposite sides of Hg²⁺. As a result, Hg²⁺ is positioned along the symmetry axis and with the Hg—N and Hg—S distances nearly identical for both conformers, while the S—S distance increases to respective 4.93 and 5.22 Å. The cis conformer is calculated to be 0.07 eV higher in energy than the trans one, so both conformers may co-exist. The potential energy barrier stabilizing the cis conformer is evaluated as 0.09 eV high and corresponds to one S-containing ring being co-planar with the central ring. In order to check the possibility of an alternative, asymmetric Hg—C bonding suggested for a similar system in a paper, (see A. K. Shigemoto, C. N. Virca, S. J. Underwood, L. R. Shetterly and T. M. McCormick, J. Coord. Chem., 2016, 69, 2081-2089) additional calculations have been carried out with one of the S-carrying rings rotated around the C—C bond connecting it to the central ring, so that S points away from Hg. Accordingly, to enable the Hg—C bonding, the proton nearest to Hg has been transferred to this atom or removed altogether. As a result, the energy of the system has increased by about 3 eV in the former or a few eV still higher in the latter case, leading us to discard such interactions in our case. Chemical titration (Jobs Plot) confirms 1:1 L:Hg(II) stoichiometry of the complex (see FIG. 2D).

### Design of Hg(II) Sensing/Removing Nanomaterial

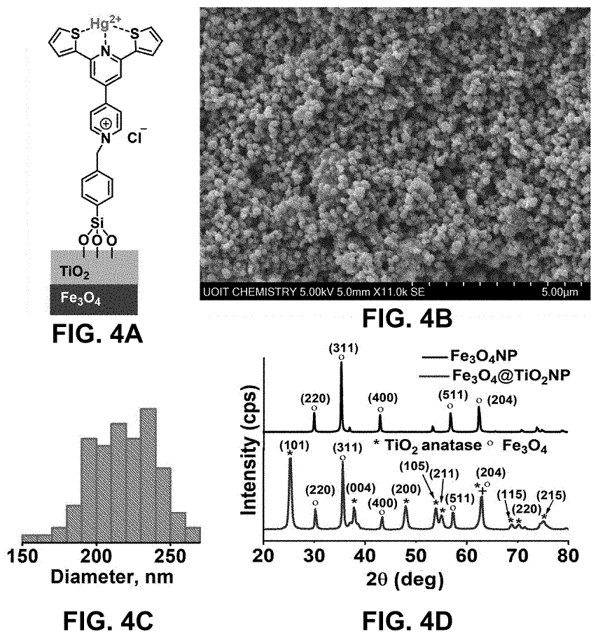

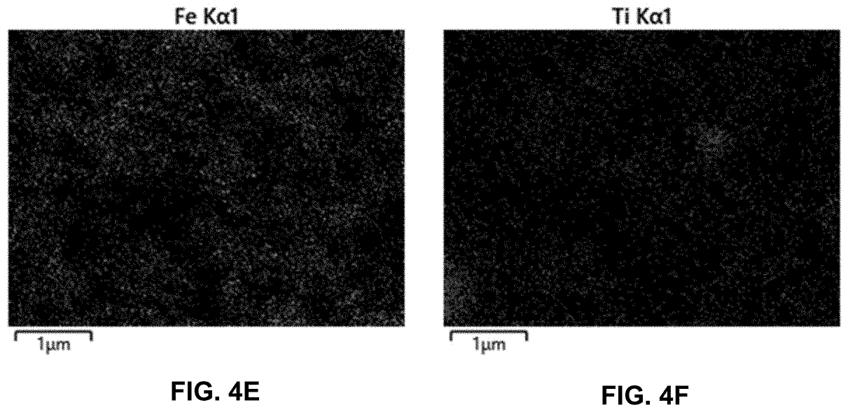

In accordance with the teachings herein, at least one embodiment of the nanomaterial that is able to detect and remove Hg(II) comprises the formation of magnetic core-shell Fe₃O₄@TiO₂ nanospheres, with pre-functionalization provided by a templating chlorobenzylsiloxane layer, and covalent anchoring of ligand L on the surface support by selective quaternization of a non-chelating pyridinic nitrogen atom (FIG. 4A). This approach allows the dispersion of nanospheres in contaminated solution and easy separation of reacted material using the external magnetic force. Since larger Fe₃O₄ nanoparticles demonstrate better magnetic properties, the diameter of core NPs may be selected to exceed 100 nm for the best magnetic separation. Therefore, ferrite microspheres of an average diameter of 200 nm were targeted. Magnetic core-shell Fe₃O₄@TiO₂ nanospheres were synthesized by coating Fe₃O₄ core by mesoporous nanocrystalline titania via hydrothermal method as previously reported (see for example Ma, W.-F.; Zhang, Y.; Li, L.-L.; You, L.-J.; Zhang, P.; Zhang, Y.-T.; Li, J.-M.; Yu, M.; Guo, J.; Lu, H.-J.; Wang, C.-C., Tailor—ACS Nano 2012, 6 (4), 3179-3188).

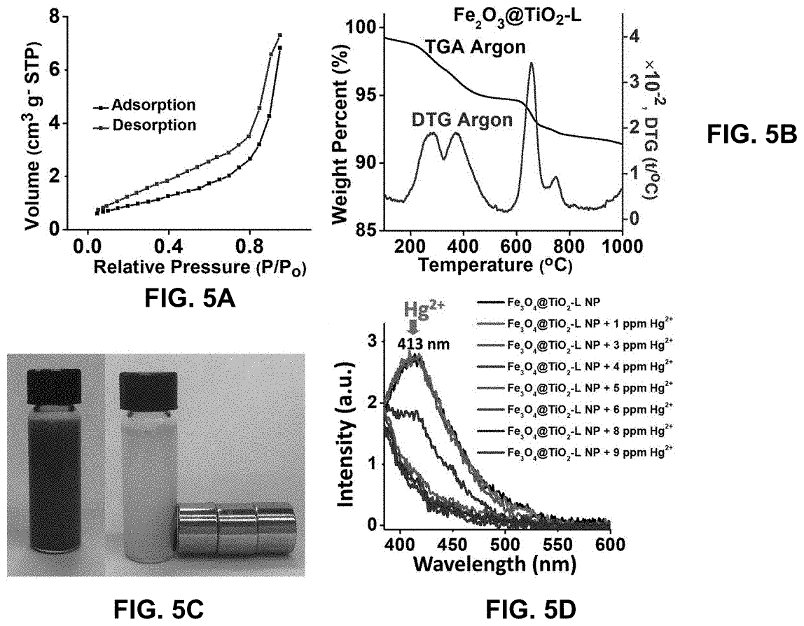

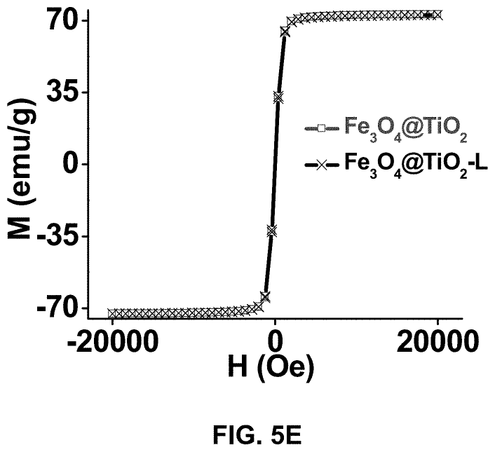

SEM analysis confirms the formation of spherical features of the desired size with narrow size distribution (see FIGS. 4B and 4C). Formation of the core-shell structure was supported by XRD showing the presence of both characteristic peaks of Fe₃O₄ and TiO₂. Thus, six characteristic peaks for the typical cubic structure of Fe₃O₄: (220), (311), (400), (422), (511), (204) (according to JCPDS 19-629) are sharp and intense indicating well defined crystalline core of Fe₃O₄. In addition, set of peaks characteristic for TiO₂ anatase phase (101), (004), (200), (105), (211), (115), (220), (215) (according to JCPDS 21-1272) is clearly visible (see FIG. 4D). The average size of TiO₂ crystallites in the shell of the material was calculated from the broadening of the (101) reflection using Scherrer's formula (see A. L. Patterson, Phys. Rev., 1939, 56, 978-982) and determined to be 15.3 nm, which is consistent with previously published values. Energy dispersive X-ray (SEM-EDX) mapping of Fe₃O₄@TiO₂ shows a uniform distribution of both iron and titanium within the material indicating a smooth and homogenous coating of magnetite core with a TiO₂ layer (FIGS. 4E and 4F). The surface area of the Fe₃O₄@TiO₂ performed by the Brunauer-Emmett-Teller (BET) method using nitrogen adsorption-desirption (FIG. 5A) was determined to be 58.26 m²/g with the average pore radius of 2.14 nm, which is enough for the in-pore molecular deposition. It was also found that further material functionalization by L using straight-forward siloxane chemistry results in desired Fe₃O₄@TiO₂-L material.

The thermogravimetric analysis (TGA) of Fe₃O₄@TiO₂-L performed under argon (FIG. 5B) indicates that the material is stable at temperatures up to 200° C. In the 220-450° C. temperature range, chemically-attached L start decomposing via a two-step weight loss process as previously described for a similar ligand (see J. T. S. Allan, S. Quaranta, I. I. Ebralidze, J. G. Egan, J. Poisson, N. O. Laschuk, F. Gaspari, E. B. Easton and O. V. Zenkina, ACS Appl. Mater. Interfaces, 2017, 9, 40438-40445). Finally, the mass loss at 600-800° C. is associated with the decomposition of the siloxane templating layer (see W.-J. Wu, J. Wang, M. Chen, D.-J. Qian and M. Liu, J. Phys. Chem. C, 2017, 121, 2234-2242).

### Properties of Mercury Uptake Material

Fe₃O₄@TiO₂-L material demonstrates significant magnetic saturation of 70 emu/g that allows easy separation of dispersed material from acetonitrile or aqueous solutions by application of an external magnetic field (FIGS. 5C and 5E). The material may be easily re-dispersed in the media by simply shaking the sample. Indeed, agitating Fe₃O₄@TiO₂-L in aqueous solutions of Hg(II) followed by the removal of the material by a magnet, results in the decrease of Hg(II) concentration in the solution. The absorption capacity of 13.35 μg of Hg²⁺ per one mg of Fe₃O₄@TiO₂-L material was determined using cold vapor atomic absorption (AA) technique.

The ability of Fe₃O₄@TiO₂-L to uptake Hg(II) from the acetonitrile solutions can be directly observed using fluorescence spectrometry (see FIG. 5D). In contrast to L, the emission intensity at 413 nm of the material is significantly lower. DFT modelling of surface-attached L confirms the excited states start from lower energies (about 2 eV) and have low f values (0.01 and less) up to 4 eV excitation. This may explain the strong (by 2 orders of magnitude) reduction of the intensity of the fluorescence band. In this example embodiment if a mercury detecting material in accordance with the teachings herein, a maximum absorbing/sensing capacity of the material is reached upon the reaction of 1 mg of nanomaterial to 3 mL of acetonitrile solution containing 5 ppm (5 μg/mL) of Hg(II). This corresponds to 15 μg of Hg²⁺ absorbed by 1 mg of the material, consistent with the absorption capacity of the material introduced to aqueous solutions of Hg(II). LOD for the “turn-off” peak of Fe₃O₄@TiO₂-L at 413 nm (λexc=380) is 2.67 ppm of Hg²⁺.

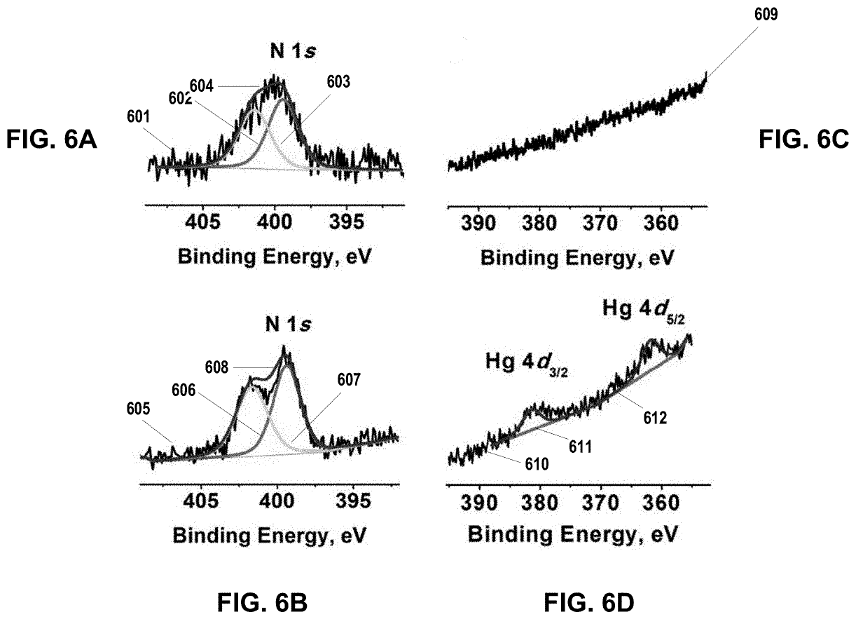

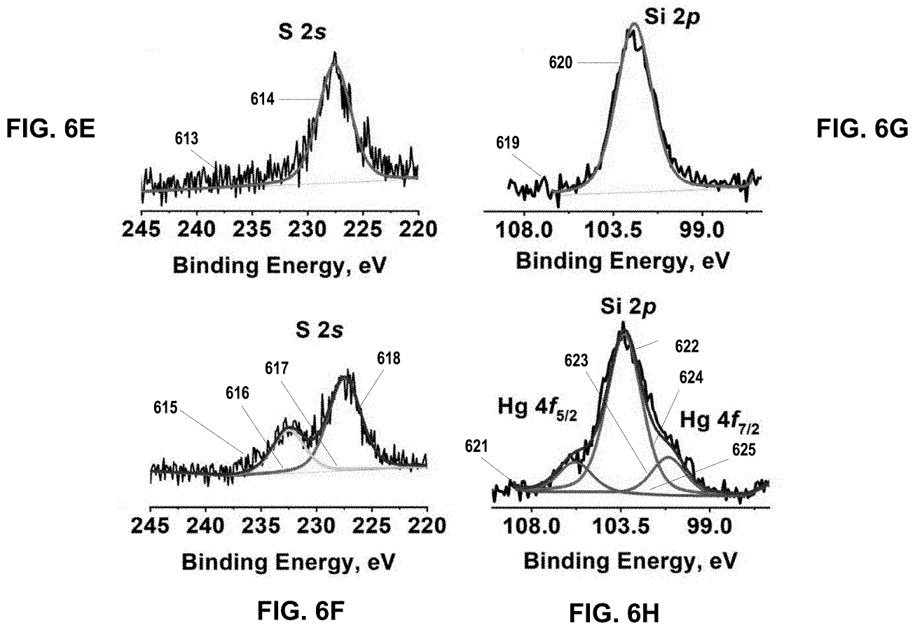

XPS is a powerful tool for structural and electronic characterization of monolayer-based nanoarchitectures. The efficiency of the Fe₃O₄@TiO₂-L system in Hg(II) uptake can be investigated by determining XPS Hg:N ratio. However, the XPS analysis of materials containing silicon and mercury is not trivial. FIGS. 6A-6H show x-ray photoelectron spectra of Fe₃O₄@TiO₂-L and Fe₃O₄@TiO₂-L-Hg²⁺. Curves 601, 605, 609, 610, 613, 615, 619, and 621 show experimental data. Curves 602, 603, 604, 606, 607, 608, 611, 612, 614, 616, 617, 618, 620, 622 and 623 show overall fitted spectra. Curve 624 represents silicon from the silane template and curve 625 corresponds to the Hg²⁺. The most intense mercury (Hg 4f) peaks overlap with the most intense peak of silicon (Si 2p) located at 103.3 eV. The peak deconvolution is possible (see FIG. 6H) if fixing a full width at half-maximum (fwhm) of Si 2p peak at the same level as Si 2p peak of starting (non-contaminated by mercury) Fe₃O₄@TiO₂-L material (see FIG. 6G). Peak area normalization between N is and Hg 4f using relative XPS sensitivity factors as determined by Wagner (see C. D. Wagner, L. E. Davis, M. V. Zeller, J. A. Taylor, R. H. Raymond and L. H. Gale, Surf. Interface Anal., 1981, 3, 211-225) gives an N/Hg ratio equal to 2.0:0.38. This suggests that in this case, only 38% of surface-anchored L molecules form the complex.

The complex formation is also confirmed by the splitting of the S 2s peak. The S 2s peak of Fe₃O₄@TiO₂-L is centered at 227.5 eV, which is characteristic to S²⁻ in thio-organic compounds (see M. A. Hanif, I. I. Ebralidze and J. H. Horton, Appl. Surf. Sci., 2013, 280, 836-844). When the material is reacted with mercury, a new S 2s peak at 232.3 eV appears demonstrating that the electron density is withdrawn from sulfur perhaps through the σ-bonding to complexed mercury. This is in a good agreement with DFT calculations (vide supra) demonstrating larger shared electron numbers and larger values of Wiberg bond index for Hg—S as compared to Hg—N. The ratio of the newly formed to the initial S 2s peak is 1.0:1.6, which gives 37% of sulfur involved in the complex formation. The XPS area of N 1s contains 2 peaks: one corresponding to a nitrogen atom in the chelating bis-thienylpyridine moiety at 399.3 eV and the second one is characteristic to N⁺ of the anchoring pyridyl unit at 401.6 eV. Interestingly, the complex formation has a minor influence on the positions of N1s peaks. Finally, the appearance of the secondary mercury line (Hg 4d) signals that are not overlapping with any of the materials elements (see FIG. 6D) directly demonstrates mercury uptake and formation of Fe₃O₄@TiO₂-L-Hg²⁺. The Hg 4d_{5/2} peak is centered at 361.9 eV while Hg 4d_{5/2} peak is located at 381.6 eV. While Hg 4d peaks were recently reported for some crystalline inorganic compounds, (T. V. Vu, A. A. Lavrentyev, B. V. Gabrelian, O. V. Parasyuk, V. A. Ocheretova and O. Y. Khyzhun, J. Alloys Compd., 2018, 732, 372-384) to the best of the inventors' knowledge, this is the first report on Hg 4d peaks for a surface-anchored metal complex.

The fact that not all of the L molecules deposited on the surface form the complex with Hg(II) can be explained by the close-packed monolayer of L and large size of Hg(II).

Interference Studies of the Mercury Uptake by the Material Fe₃O₄@TiO₂-L

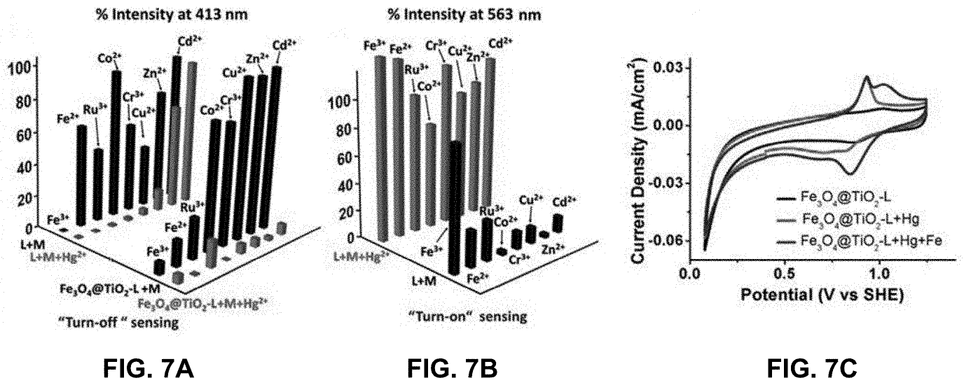

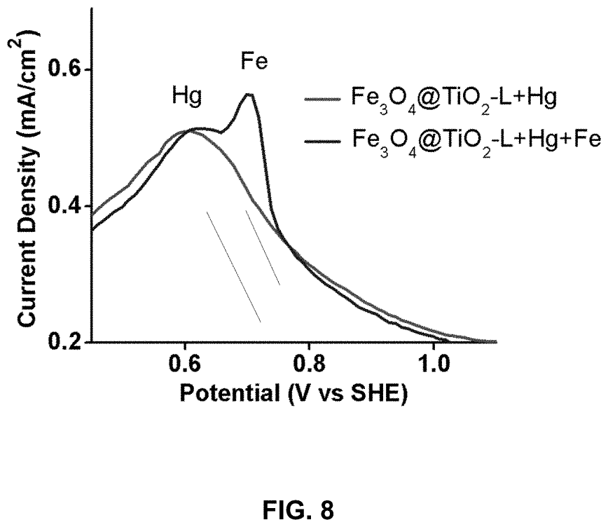

To study the interference, the response of L and Fe₃O₄@TiO₂-L to Hg(II) in the presence of various metal ions was explored. The analysis of “turn off” (excitation at 330 nm, emission at 413 nm) and “turn on” (excitation at 385 nm, emission at 563 nm) fluorescence emission peaks of L reacted with metal ions (see FIGS. 7A-7C) allows easy recognition of Hg(II). Thus, the intensity of the “turn off” emission band of L solution in acetonitrile undergoes just minor changes in the presence of Hg²⁺—Zn²⁺ and Hg²⁺—Cd²⁺ binary mixtures. This interference is consistent with literature reports that claim significant affinity of terpyridine-based derivatives to Cd²⁺ and Zn²⁺ (N. O. Laschuk, I. I. Ebralidze, D. Spasyuk and O. V. Zenkina, Eur. J. Inorg. Chem., 2016, 22, 3530-3535. Y. Hong, S. Chen, C. W. T. Leung, J. W. Y. Lam, J. Liu, N.-W. Tseng, R. T. K. Kwok, Y. Yu, Z. Wang and B. Z. Tang, ACS Appl. Mater. Interfaces, 2011, 3, 3411-3418). However when L is reacted with Hg²⁺, a significant increase of corresponding “turn-on” band is almost unaffected by the presence either Cd²⁺, or Zn²⁺. Moreover, mercury-free solutions of Cd²⁺ and Zn²⁺ have a minor influence on the “turn-on” band. In contrast, Fe³⁺ was found to be the main interference factor. Interference with iron ions is a common feature of many reported molecular receptors for mercury detection (Lv, H.; Ren, Z.; Liu, H.; Zhang, G.; He, H.; Zhang, X.; Wang, S., The Turn-Off Fluorescent Sensors Based on Thioether-Linked Bisbenzamide for Fe3+ and Hg²⁺. Tetrahedron 2018, 74 (14), 1668-1680). Discovering the influence of potentially competitive ions on the Hg (II) detection by Fe₃O₄@TiO₂-L demonstrates a significant interference with Fe^{2+/3+} and Ru³⁺ ions, while no to negligible interference was observed for Zn²⁺, Cd²⁺, Cu²⁺, Cr³⁺, and Co²⁺. This difference in selectivity of the material compared to L may be explained by significant changes in electronics of L upon quaternization step performed to anchor the molecule to the chlorobenzylsiloxane pre-modified surface, as previously reported for other ligand architectures. [N. O. Laschuk, I. I. Ebralidze, J. Poisson, J. G. Egan, S. Quaranta, J. T. S. Allan, H. Cusden, F. Gaspari, F. Y. Naumkin, E. B. Easton and O. V. Zenkina, ACS Appl. Mater. Interfaces, 2018, 10, 35334-35343.] In order to distinguish if the fluorometric “turn-off” drop of the Fe₃O₄@TiO₂-L material is caused by the mercury or iron uptake, the reacted material can be deposited on glassy carbon electrode. Both cyclic voltammetry (CV) and differential pulse voltammetry (DPV) allow discrimination between mercury and iron peaks (FIG. 8).

Probing Ligands L₁-L₃ for Hg²⁺ Sensing

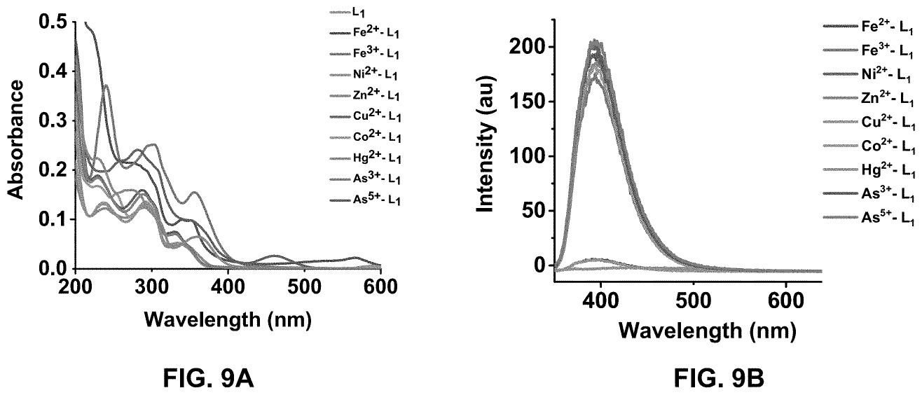

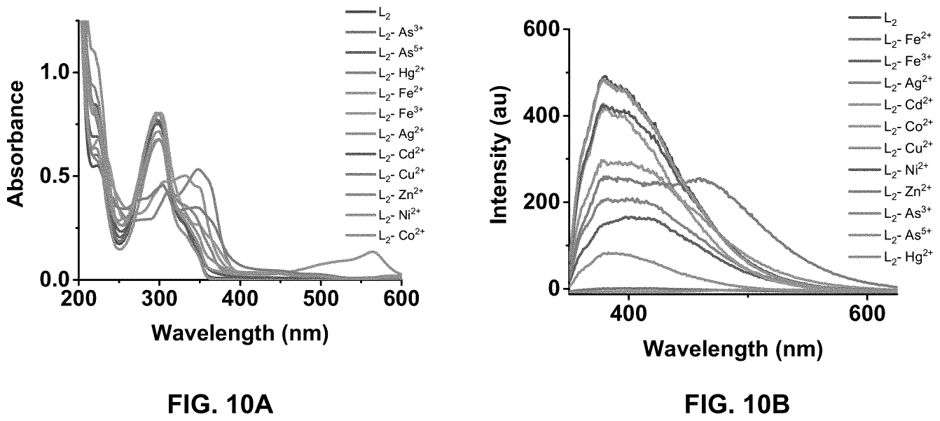

Ligands L₁, L₂, and L₃ demonstrate significant Hg²⁺ affinity and can be used alone or as building blocks of materials for Hg²⁺ sensing and removal. The analysis of UV-vis and fluorescence outputs of L₁ and L₂ are shown in FIGS. 9A-9B and 10A-10B. An analysis of fluorescence turn off emission peak for L₃ in the presence of different metal ions is shown in FIG. 11.

### Schemes

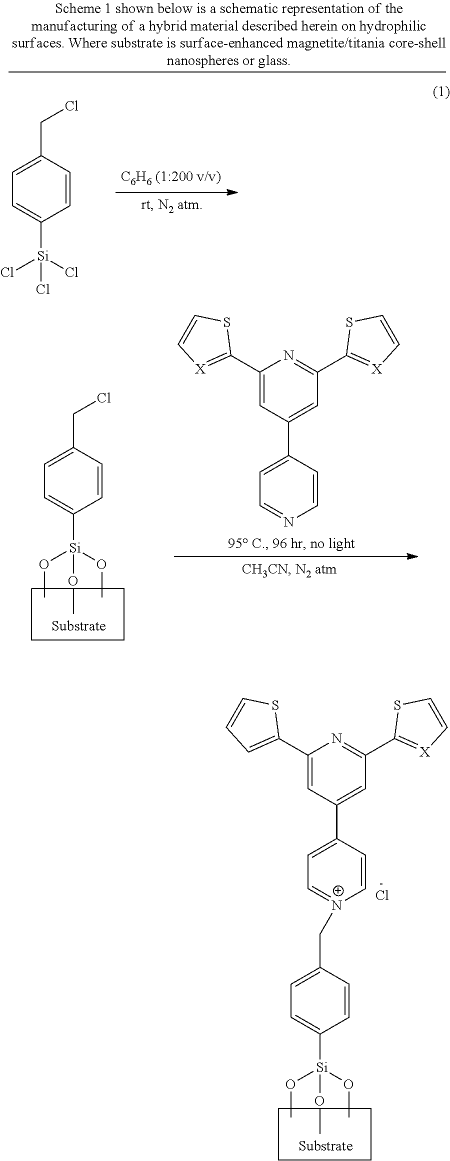

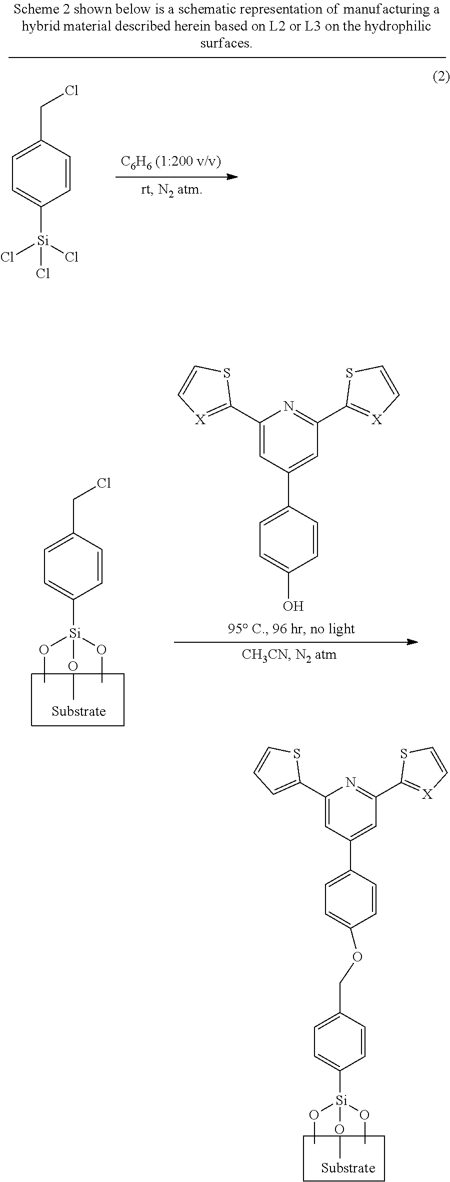

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

1. A composite material for the detection of an analyte, the composite material comprising:
a ligand-functionalized monolayer; and
a support material coupled to the ligand-functionalized monolayer;
wherein the ligand-functionalized monolayer includes one or more ligands having the formula: 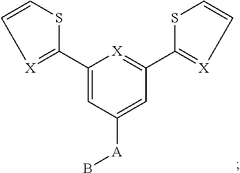
wherein A comprises a linear or a cyclic aliphatic moiety, an aromatic ring or a fused aromatic ring system, a heteroaromatic ring, a fused heteroaromatic ring system, quaternary ammonium salt, or a combination thereof;
B comprises hydrogen or a chemically derivatizable group such as alkene, alkyne, amino acid, azide, phosphate, phosphonate, carboxyl group, silane, siloxane, sulfate, quaternary ammonium salt, thiol, alkyl thiol, or thioester; and
X comprises carbon, nitrogen, sulphur or oxygen.

2. The composite material of claim 1, wherein X is nitrogen.

3. The composite material of claim 1, wherein A is pyridine.

4. The composite material of claim 1, wherein the ligand-functionalized monolayer includes one or more ligands having the formula:

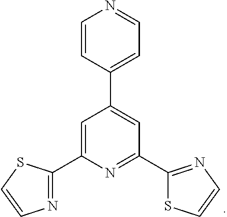 [SMILES: c1cc(-c2cc(-c3nccs3)nc(-c3nccs3)c2)ccn1]

5. The composite material of claim 1, wherein A is benzene.

6. The composite material of claim 1, wherein A-B is phenol.

7. The composite material of claim 1, wherein the ligand-functionalized monolayer includes one or more ligands having the formula:

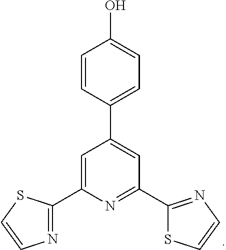 [SMILES: Oc1ccc(-c2cc(-c3nccs3)nc(-c3nccs3)c2)cc1]

8. The composite material of claim 1, wherein the analyte is mercury.

9. A composite material for the detection of an analyte, the composite material comprising:
a ligand-functionalized monolayer; and
a support material coupled to the ligand-functionalized monolayer;
wherein the ligand-functionalized monolayer includes one or more ligands having the formula: 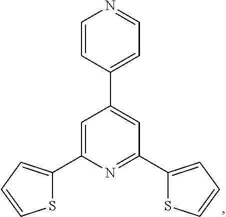 [SMILES: c1csc(-c2cc(-c3ccncc3)cc(-c3cccs3)n2)c1]
or the formula:

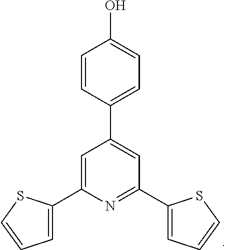 [SMILES: Oc1ccc(-c2cc(-c3cccs3)nc(-c3cccs3)c2)cc1]

10. The composite material of claim 9, wherein the composite material undergoes a fluorescence change in the presence of one or more target analytes.

11. The composite material of claim 10, wherein the fluorescence change is a quenching of fluorescence at a specific wavelength.

12. The composite material of claim 11, wherein the specific wavelength is between 300 nm and 600 nm.

13. The composite material of claim 10, wherein the target analytes include mercury.

14. The composite material of claim 12, wherein the ligand-functionalized monolayer includes TiO₂.

15. The composite material of claim 9, wherein the support material includes a nanoparticle.

16. The composite material of claim 15, wherein the nanoparticle is a Fe₃O₄ magnetic nanoparticle.

17. A method for the fluorescence detection of mercury, the method comprising:
providing a fluorescence sensing indicator comprising the composite material of claim 1;
exposing the indicator to a source of mercury; and
detecting any fluorescence changes.

18. The method of claim 17, wherein providing the fluorescence sensing indicator includes providing a fluorescence sensing indicator having a characteristic fluorescence wavelength and detecting any fluorescence changes includes detecting any fluorescence changes includes detecting a quenching of fluorescence at a characteristic wavelength.

19. The method of claim 18, wherein detecting a quenching of fluorescence at a characteristic wavelength includes detecting a quenching of fluorescence at a wavelength between 300 nm and 600 nm.

20. A method of removing mercury from a solution, the method comprising:
providing the composite material of claim 1 to the solution containing mercury, the composite material being magnetic; and
applying a magnetic field to the solution to remove the composite material and at least a portion of mercury from the solution.
